(19) United States
(12) Patent Application Publication
(10) Pub. No.: US 2021/0228260 A1
(43) Pub. Date: Jul. 29, 2021

(54) CUSTOMIZED WAVEFORM AND CONTROL FOR PULSED ELECTRIC FIELD ABLATION SYSTEMS

(71) Applicant: BOSTON SCIENTIFIC SCIMED, INC., MAPLE GROVE, MN (US)

(72) Inventors: LARRY D. CANADY, JR., HAM LAKE, MN (US); BRUCE R. FORSYTH, HANOVER, MN (US); HONG CAO, MAPLE GROVE, MN (US); TIMOTHY A. OSTROOT, COKATO, MN (US); JONATHAN TYLER GORZYCKI, BLAINE, MN (US)

(73) Assignee: BOSTON SCIENTIFIC SCIMED, INC., MAPLE GROVE, MN (US)

(21) Appl. No.: 17/129,043

(22) Filed: Dec. 21, 2020

Related U.S. Application Data
(60) Provisional application No. 62/966,920, filed on Jan. 28, 2020.

(51) Int. Cl. A61B 18/12

(52) U.S. Cl. CPC A61B 18/1206; A61B 2018/00577

(57) ABSTRACT

Systems and methods for performing and controlling ablation therapy. Examples provide adaptive therapy outputs that allow a user to select among various feedback parameters, parameter limits, and therapy profiles, to be implemented by an ablation system. The ablation system adaptively issues therapy by monitoring one or more feedback parameters to determine changes to make to therapy outputs.

### CROSS REFERENCE TO RELATED APPLICATIONS

This application claims the benefit of and priority to U.S. Provisional patent Application Ser. No. 62/966,920 filed on Jan. 28, 2020, the disclosure of which is incorporated herein by reference.

### BACKGROUND

Removal or destruction of diseased tissue is a goal of many cancer treatment methods. Tumors may be surgically removed, however, less invasive approaches garner much attention. Tissue ablation is a minimally invasive method of destroying undesirable tissue in the body. A variety of ablation techniques have been developed, many using the application of electricity or other energy via a probe placed on or inserted into or adjacent target tissue. For example, heat-based thermal ablation adds heat to destroy tissue. Radio-frequency (RF), microwave and high intensity focused ultrasound ablation can each be used to raise localized tissue temperatures well above the body's normal 37 degrees C.

Irreversible electroporation (IRE) uses electric fields to expand pores in the cell membrane beyond the point of recovery, causing cell death for want of a patent cell membrane. The spatial characteristics of the applied field control which cells and tissue will be affected, allowing for better selectivity in the treatment zone than with thermal techniques. IRE typically uses a narrower pulse width than RF ablation to reduce thermal effects.

Various forms of feedback are used in ablation systems. For example, some robotic and cardiac ablation systems use visualization or electrical field measurements to monitor probe position. A cardiac ablation system may use an electrode array in the form of a basket placed against the interior of a heart chamber to electrically sense and target the location of aberrant electrical activity in the myocardium and to monitor the position of an ablation probe relative to the basket and targeted tissue. Visualization, such as by fluoroscopy, may be used to identify probe position. Some systems, particularly robotic systems, may use force or other sensors to detect when tissue is contacted with a probe. Still other systems, such as thermal ablation systems, monitor temperature of select tissue to ensure adequate temperature to cause cell destruction while limiting maximum temperatures to avoid destruction of non-target tissue. Some literature suggests the use of tissue impedance to monitor the status of ablation; as cells are destroyed, fluid within cell membranes escapes, reducing the local impedance, providing a marker of ablation progress. Such feedback approaches have provided valuable information to manage device or probe position, to manage power level, and/or to determine therapy success or completion. However, greater integration of these feedback mechanisms for additional and alternative control methods are still desired.

### Overview

The present inventors have recognized, among other things, that a problem to be solved is the need for new and/or alternative planning and control methods and systems for ablation purposes.

A first illustrative, non-limiting example takes the form of a system for controlling an ablation therapy comprising: a signal generator adapted to provide electrical output for ablation therapy; and a user interface operatively linked to the signal generator, the user interface configured to interact with a user by: providing the user a list of available closed loop control parameters to select from; receiving from the user a selection of one or more closed loop control parameters; and for at least one user selected closed loop parameter, presenting the user with an input screen for selecting or approving one or more limits for the user selected closed loop parameter; further wherein the signal generator is configured to deliver a therapy regimen as follows: generating a first electrical output having a first output parameter set; sensing a signal related to the user selected closed loop parameter and comparing the sensed signal to the user selected or approved limit for the user selected closed loop parameter; adjusting the first output parameter set to create a second output parameter set; and generating a second electrical output using the second output parameter set, wherein at least the second output parameter set is configured for ablating tissue.

Additionally or alternatively, the closed loop control parameters comprise at least user selectable options for phase. Additionally or alternatively, the closed loop control parameters comprise at least user selectable options for reactive impedance. Additionally or alternatively, the closed loop control parameters comprise at least user selectable options for inter-pulse or inter-burst voltage. Additionally or alternatively, the closed loop control parameters comprise at least user selectable options for multi-path impedance. Additionally or alternatively, the therapy regimen comprises a plurality of bursts each comprising a plurality of pulses, wherein the first and second electrical outputs occur within the same pulse. Additionally or alternatively, the therapy regimen comprises a plurality of bursts each comprising a plurality of pulses, wherein the first and second electrical outputs occur within separate pulses of the same burst. Some examples may have the first and second electrical outputs occur within the same pulse or within separate pulses within a burst. Additionally or alternatively, the first and second electrical outputs differ from one another in terms of electrodes selected as anodes or cathodes for each of the outputs. Additionally or alternatively, the first and second electrical outputs differ from one another in terms of slew rate.

A second illustrative and non-limiting example takes the form of a system for controlling an ablation therapy comprising: a signal generator adapted to provide electrical output for ablation therapy; and a user interface operatively linked to the signal generator, the user interface configured to interact with a user by: providing the user a list of available therapy profiles to select from; and receiving from the user a selection of one of the available therapy profiles; further wherein the signal generator is configured to deliver a therapy regimen as follows: configuring a first output therapy parameter set using the selected therapy profile; generating one or more first therapy outputs using the first output therapy parameter set; sensing one or more first feedback parameters; comparing the first feedback parameters to an expected feedback parameter to generate one or more first comparison results, wherein the expected feedback parameter is associated with the selected therapy profile; and configuring a second output therapy parameter set using the first comparison results.

Additionally or alternatively, the first feedback parameters and the expected feedback parameter relate to a relative change in impedance. Additionally or alternatively, the first feedback parameters and the expected feedback parameter relate to a change in sensed phase. Additionally or alternatively, the first feedback parameters and the expected feedback parameter relate to inter-pulse or inter-burst voltage. Additionally or alternatively, the first feedback parameters and the expected feedback parameter relate to multi-path impedance. Additionally or alternatively, the therapy regimen comprises a plurality of bursts each comprising a plurality of pulses, wherein the first and second electrical outputs occur within the same pulse. Additionally or alternatively, the therapy profiles comprise a schedule of therapy output parameters to use during the duration of the therapy regimen, wherein the signal generator is adapted to configure the second output therapy parameter set as follows: if the first feedback parameters correlate with the expected feedback parameters, using the schedule of therapy output parameters for the selected therapy profile to define the second output therapy parameter set; or if the first feedback parameters do not correlate with the expected feedback parameters, modifying the schedule of therapy output parameters for the selected therapy profile in response to the first feedback parameters.

Additionally or alternatively, the therapy regimen comprises a plurality of bursts each comprising a plurality of pulses, wherein the first and second electrical outputs occur within separate pulses of the same burst. Additionally or alternatively, the first and second electrical outputs differ from one another in terms of electrodes selected as anodes or cathodes for each of the outputs. Additionally or alternatively, the first and second electrical outputs differ from one another in terms of slew rate.

A third illustrative and non-limiting example takes the form of a system for controlling an ablation therapy comprising: a signal generator adapted to provide electrical output for ablation therapy; and a user interface operatively linked to the signal generator, the user interface configured to interact with a user by: providing the user a list of available therapy profiles to select from; and receiving from the user a selection of one of the available therapy profiles; further wherein the signal generator is configured to deliver a therapy regimen as follows: defining, for the selected therapy profile, at least a portion of a therapy regimen comprising a plurality of bursts of pulses, each burst comprising a plurality of pulses, each pulse comprising a plurality of pulse segments; configuring a first output therapy parameter set using the selected available therapy profile, the first output therapy parameter set defining a first predetermined segment of a predetermined pulse of a predetermined burst; generating the first predetermined segment of using the first output therapy parameters; sensing one or more first feedback parameters; using the sensed first feedback parameters to define a second predetermined segment occurring after the first predetermined segment in the predetermined pulse of the predetermined burst.

This overview is intended to provide an introduction to the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an approximation of different therapy modalities associated with a combination of electrical field strength and pulse duration;

FIG. 2 shows an illustrative ablation system in use;

FIGS. 3A-3C show illustrative ablation waveforms;

FIGS. 4A-4C illustrate user interface elements for select examples;

FIGS. 5-6 illustrate therapy application using a multiple electrode configuration;

FIGS. 7-9 show illustrative methods in block form; and

FIG. 10 is a block diagram for an illustrative ablation system.

### DETAILED DESCRIPTION

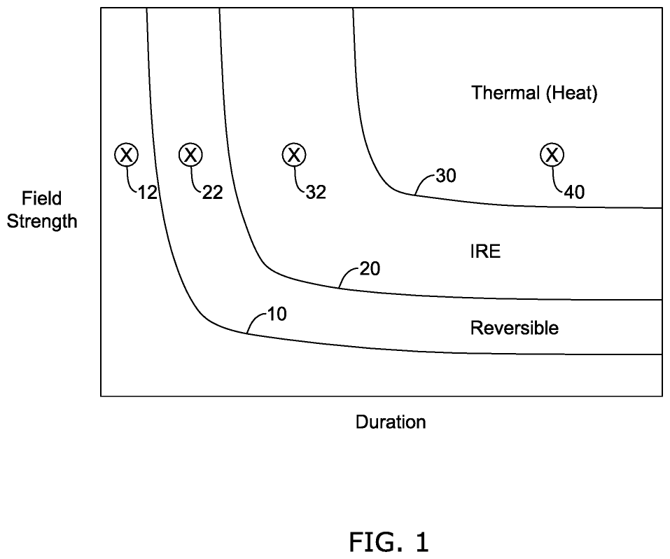

FIG. 1 shows an approximation of different biophysical responses dependent on the amplitude-time relationship of delivered electrical pulses. The thresholds between cellular responses (10, 20, 30) operate generally as a function of the applied field strength and pulse duration. Below a first threshold 10, no effect occurs; between the first threshold 10 and a second threshold 20, reversible electroporation occurs. When reversible electroporation occurs, the cellular membrane opens, allowing, for example, entry or exit of particles that might otherwise be kept in or out by the cellular membrane, but the effect is reversible following termination of the applied field, as the cell membrane returns to an intact state.

Above the second threshold 20, and below a third threshold 30, primarily irreversible electroporation (IRE) occurs. With IRE, the cell membrane forms openings as with reversible electroporation, but the quantity and/or size of the openings is sufficient to cause failure of the cell membrane, which cannot recover, leading to cell death.

Above a third threshold 30, the effects begin to be primarily thermal, driven by tissue heating. Thus, for example, at a given field strength and duration there may be no effect (location 12), and extending the duration of the field application can yield reversible electroporation (location 22), IRE (location 32), and thermal ablation (location 40).

As described in U.S. Pat. No. 6,010,613, a transmembrane potential in the range of about one volt is needed to cause reversible electroporation, however the relationship between pulse parameters such as timing and duration and the transmembrane potential required for reversible electroporation remains an actively investigated subject. The required field may vary depending on characteristics of the cells to be treated. At a macro level, reversible electroporation requires a voltage in the level of hundreds of volts per centimeter, with IRE requiring a still higher voltage. As an example, when considering in vivo electroporation of liver tissue, the reversible electroporation threshold field strength may be about 360 V/cm, and the IRE threshold field strength may be about 680 V/cm, as described in U.S. Pat. No. 8,048,067. Generally speaking, a plurality of individual pulses are delivered to obtain such effects across the majority of treated tissue; for example, 2, 4, 8, 16, or more pulses may be delivered. Some examples may deliver hundreds of pulses.

The electrical field for electroporation has typically been applied by delivering a series of individual pulses each having a duration in the range of one to hundreds of microseconds. For example, U.S. Pat. No. 8,048,067 describes analysis and experiments performed to illustrate that the area between lines 20 and 30 in FIG. 1 actually exists, and that a non-thermal IRE therapy can be achieved, using in several experiments a series of eight 100 microsecond pulses delivered at 1 second intervals.

The tissue membrane does not return instantaneously from a porated state to rest. As a result, the application of pulses close together in time can have a cumulative effect as described, for example, in U.S. Pat. No. 8,926,606. In addition, a series of pulses can be used to first porate a cell membrane and then move large molecules through generated, reversible pores, as described in US PG Patent App. Pub No. 2007/0025919.

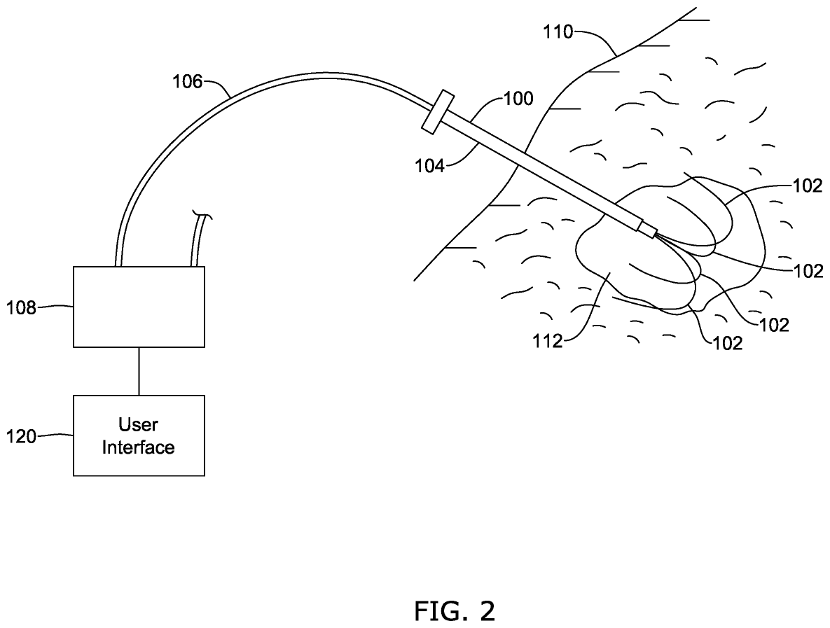

FIG. 2 shows a prior art LeVeen® needle as part of an overall system for ablation therapy delivery. As described in U.S. Pat. No. 5,855,576, the device comprises an insertable portion 100 having a shaft 104 that extends to a plurality of tissue piercing electrodes 102 that can be extended or retracted once a target tissue 112 of a patient 110 is accessed. The proximal end of the apparatus is coupled by an electrical connection 106 to a power supply 108, which can be used to supply RF energy. A user interface 120 is provided to allow a physician or other user to control the activities of the power supply 108, in conjunction with physical control over the probe 100.

As originally implemented, the LeVeen® needle would be used to deliver thermal ablation to the target tissue. For example, as described in the '576 patent, a return electrode in the form of a plate or plates may be provided on the patient's skin, a return electrode could be provided as another tissue piercing electrode, or a return electrode may be provided on the shaft 104 near its distal end, proximal of the tissue piercing electrodes 102. An RF signal would be applied to cause heating of the target tissue, effecting thermal ablation.

Enhancements on this probe design can be found, for example, in U.S. Pat. No. 6,638,277, which discusses independent actuation of the tissue piercing electrodes 102, both in terms of movement of the electrodes as well as separately electrically activating individual ones of the electrodes. The U.S. Pat. Nos. 5,855,576 and 6,638,277 patents are incorporated herein by reference for showing various therapy delivery probes. Still further enhancements and options are described in US PG Patent App. Pub. No. 2019/0223943, the disclosure of which is incorporated herein by reference as showing various therapy delivery probes, discloses updates and enhancements on the LeVeen® needle concept, allowing flexibility in the spacing, size and selection of electrodes. While originally developed for thermal ablation purposes, such probes are also usable for other ablation modalities including IRE as well as combination methods such as electrochemotherapy, in which the electric fields are used to cause permeability of the cell membrane to allow otherwise non-permeant materials to the cell interior.

The present invention may be implemented using the LeVeen® needle and/or alternative designs described above, as well as any other suitable electrode carrying structure that enables access to the vicinity of target tissue. In some examples, the power supply 108 may include a smart adaptor, and the probe 100 and/or proximal connector 106 may comprise an electrode pattern or circuitry readable by the smart adaptor to indicate the type of probe 100 that is being used. The ablation system, in turn, may integrate information regarding the type of probe into various aspects of its operation, including to inform the available electrode combinations of the system, to aid in determining how much energy delivered will actually get to the tissue after accounting for line impedance, etc. which can reduce therapy output amplitude. The ablation system may also use such information to aid in the generation of therapy profiles, which are further discussed and explained below.

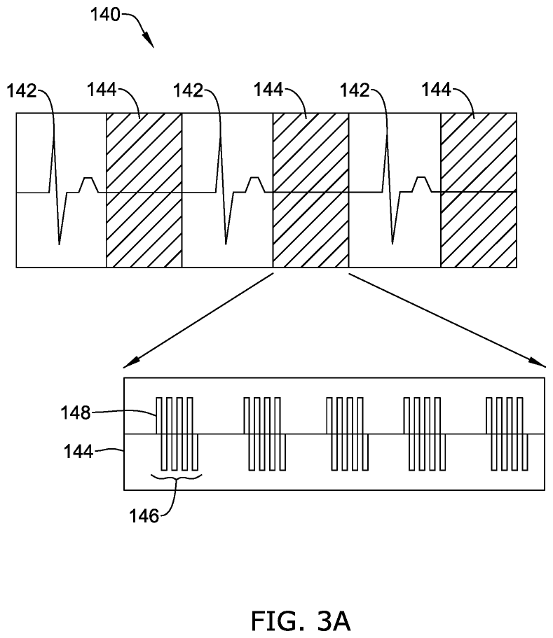

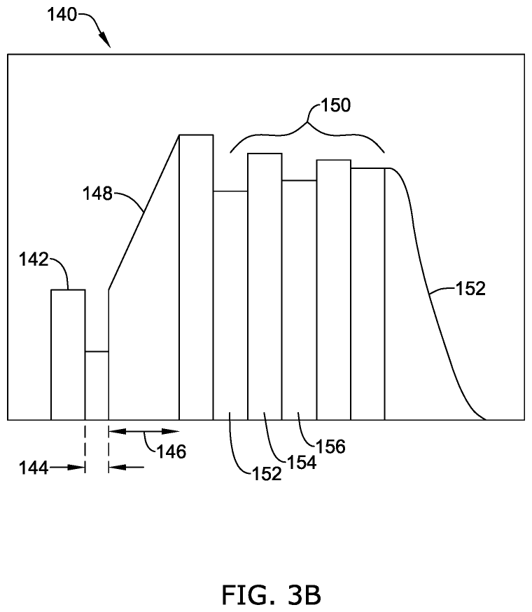

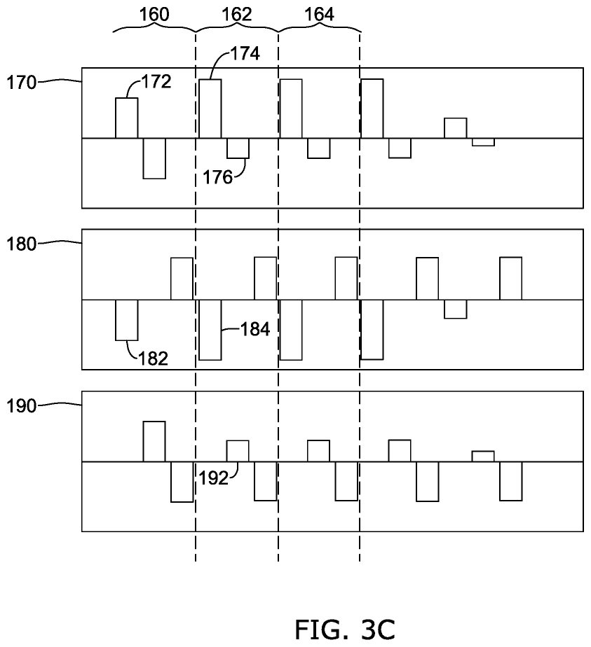

FIGS. 3A-3C show illustrative waveforms for ablation therapy. FIG. 3A shows a fairly typical approach to ablation therapy outputs. In order to avoid interfering with cardiac activity, heartbeats are sensed and used to trigger therapy output, as shown at 140. With heartbeats occurring at 142, windows for therapy delivery are defined at 144. The therapy windows 144 follow completion of a the T-wave, during which an electrical stimulus can have deleterious effects on cardiac function, such as inducing fibrillation, and end before the start of a subsequent QRS cycle; the window 144 may have a duration of tens to hundreds of milliseconds, for example. Within a therapy window 144, the ablation system may deliver therapy in the form of a series of bursts 146, each of which comprises a number of individual pulses. A typical therapy regimen will comprise a number of these bursts 146, in ranges from tens to hundreds (or more or less) bursts 144, with individual bursts comprising tens to hundreds of pulses (or more or less). An adaptive ablation therapy as used herein may modify the delivered signal at several different resolutions, including modifying therapy delivery within an individual pulse, or from one pulse to the next within a burst, or from one burst to the next within a therapy window, or from one therapy window to the next.

FIG. 3B illustrates an approach to segmented and adaptive therapy delivery within a pulse. A single pulse output is shown at 140, divided into a plurality of segments such as that shown at 142. The individual segments may be of equal duration or of different durations, such as shown at 144, 146 where the second segment of the pulse 140 is shorter than the third segment. Each pulse may have a different amplitudes and/or shape, including for example a ramped shape shown at 148, or a decaying shape at 152. The segments may be defined in advance by an algorithm or a stored set of instructions for delivery of the pulse 140. In some examples, an adaptive therapy uses data captured by sensors connected to the ablation system to determine one or more characteristics of a later segment in the pulse 140. Thus, for example, in a set of segments at 150, the system may be adjusting the output amplitude to achieve a target current or temperature in the target tissue, with adjustments of the amplitude up and down from one segment to the next. In an example, the pulse 140 may have a duration in the range of about 1 to about 1000 microseconds, or longer or shorter. Different segment shapes may be provided in a system by, for example, having a pulse delivery circuit that allows modification of slew rate or which has a fast-acting digital-to-analog conversion circuit usable to define the output.

In a specific, but non-limiting example, the pulse 140 has a duration of, for example, 10 microseconds, with a temperature sensor that can be sampled at 100 nanosecond increments, operating in a system having a microprocessor operating at over 1 gigahertz, making adjustments of segments of one microsecond or less well within the capabilities of the system. Thus, for example, a voltage output is predetermined for pulse segment 152, having a duration of 1 microsecond, and the temperature or current is sensed at the midpoint of the pulse segment and compared to a target temperature, or compared to high or low boundaries for target current, such that the voltage output can be modified for use in the subsequent pulse segment at 154. Supposing the current or temperature sensed during pulse segment 152 is below a target or setpoint, or out of a predefined current range, or is below an expected value (each of which corresponds to different examples described below), the voltage for pulse segment 154 is increased relative to pulse segment 152. The process is repeated, with measurement taking place during pulse segment 154 to determine whether to increase, decrease, or leave as-is, the voltage for pulse segment 156.

Another example of an adaptive circuit would be one in which the control management varies during delivery. In one illustration, a voltage-controlled output is generated at the start of therapy, with a transition to current-controlled therapy. For example, given a target temperature in tissue, the system may initially deliver one or more voltage defined pulses, while monitoring both current flow and temperature. Voltage manipulation may occur until a desired target temperature is achieved, at which point, the current may be noted, and subsequent pulses or pulse segments may be delivered to maintain the desired current as the impedance in the operating environment may vary. This may be useful when a system is first energized, as the local impedance may vary at start-up of the output, such that allowing a constant current output to be used runs a risk of delivering large voltages and triggering muscle stimulation distant from the therapy target or, in the alternative, that the system may not be able to establish an appropriate compliance voltage for a current-controlled output circuit until therapy has in fact begun. Once the system has delivered a few pulses or bursts, the transition to a current-controlled output may be made.

In still another example, voltage and/or current may be manipulated until a target temperature is achieved, at which time the energy delivered is noted, and subsequent pulses or pulse segments may be delivered in a manner that maintains the energy delivered while reducing pulse width. One issue that such transition in control may address is that temperature change may occur more slowly than current or voltage can be controlled. For electroporation, for example, some degree of tissue warming may be acceptable, while kept below ablation level temperatures (such as keeping temperature below or at 50 degrees C.); once a temperature limit is reached, reducing pulse width while maintaining constant energy may be a useful approach to maximizing IRE while limiting thermal effects.

FIG. 3C shows another example in a multi-channel output environment. Here, the outputs of three channels are tracked at 170, 180, 190, with each channel coupled to a separate electrode on an ablation probe. In the example, which is not intended to be limiting, outputs are delivered in triplets as indicated at 160, 162, 164, in which channel 170 delivers one pulse relative to channel 180 and one pulse relative to channel 190, with the third pulse of the triplet between channels 180 and 190, with each channel used as anode for a pulse and cathode for a pulse. Such a three-electrode rotating therapy is also described in commonly assigned U.S. Provisional Patent Applications 62/819,101, 62/819,120, and 62/819,135, the disclosures of which are incorporated herein by reference. In either a scripted or adaptive manner, the therapy pulses can be modified from one triplet to the next over time. Thus, for example, if the electrodes coupled to channels 170 and 190 are closer together, while the electrodes of channels 170 and 180 are farther apart, a constant voltage output would deliver less current and lower applied fields for the more distant electrodes; as a result, the voltage may be increased when issuing a signal between channels 170 and 180, as shown by the higher amplitude for pulse 174/184 relative to pulse 172/182. Meanwhile, the amplitude is reduced for the closer spaced electrodes, as indicated by reduced amplitude of pulses 176/192. As therapy progresses and cells are destroyed, the impedance may drop still further for certain electrode pairs and the system may react by reducing output voltage further for those electrode pairs.

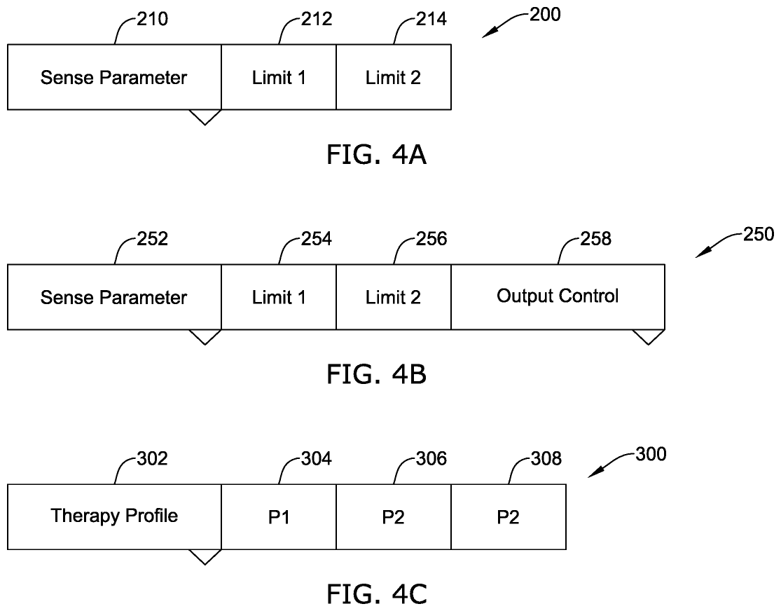

FIGS. 4A-4C illustrate user interface elements for select examples. A user interface may take the form of a touchscreen or monitor screen coupled with a mouse, keyboard or other input receiving apparatus, such as a microphone capable of receiving spoken commands. Various user interface screenshots that can be used are shown in copending U.S. Provisional Application No. 62/915,489, filed Oct. 15, 2019 and titled CONTROL SYSTEM AND USER INTERFACE FOR AN ABLATION SYSTEM, the disclosure of which is incorporated herein by reference.

In the example of FIG. 4A, a portion of a visual representation such as a screen is shown at 200. A dropdown list is user accessible at 210, in which the user can select from a list of sense parameters that the ablation system can detect, with one or more limits, such as an upper limit and lower limit, available for the selected sense parameter. Sense inputs that may be monitored include, for example and without limitation, the following feedback parameters:
Peak or average voltage, where voltage may be the voltage measured on active electrodes, or may be a field measurement taken by inactive electrodes located near active electrodes that are used to issue therapy output.
Peak or average current, which can be measured by having a current monitor on one or more output lines for the system that couple to a probe.
Pulse parameters such as pulse width and pulse delay
Peak or average energy or power, each of which may be calculated by, for example, combining voltage and current measurements and other pulse parameters; for example, power may be the voltage multiplied by current at a given point in time, while energy may be the average voltage multiplied by average current during a selected time interval.
Tissue impedance, which may be measured by monitoring current and voltage. In some particular examples different components of tissue impedance may be monitored, such as by calculating the complex impedance and monitoring real and reactive impedance separately. For example, in electroporation, permeated cell membranes allow the escape of intracellular fluid which has a higher concentration of ions than surrounding intercellular fluid, thereby reducing absolute impedance; the same mechanism will also reduce the reactive impedance as cells, which act as tiny capacitors, undergo apoptosis and become, in essence, ordinary resistors. Impedance may also be measured in a multi-path manner when there are a plurality of electrodes in the therapy area, which may be useful both to monitor spatial differences in therapy progress, as well as to select therapy outputs appropriate to the actual positioning of electrodes, which may not be precisely known when therapy is initiated or which may move during therapy due to patient motion and/or manipulation of the probe by the physician or user.
Temperature, which may be measured using a thermistor or other device or circuit having one or more characteristics that change with temperature, with temperature being measured, for example, at or near target tissue and/or at or near therapy electrodes. In some examples, an adaptive waveform is use that modifies one or more features in response to detected temperature, wherein the sensed temperature can be used in a continuous or near-continuous manner to change waveforms from one segment to the next.
Phase sensing may also be used to manage therapy outputs. The use of phase sensing may comprise, for example, comparing sensed current output to the known voltage input to determine reactive impedance in the target tissue. When the target tissue is largely filled with intact cells, the phase shift may be larger than it is when the cells become permeable due to electroporation, thus as the phase shift begins to change, therapy outputs may be modified to reduce amplitudes; if no phase shift changes are observed, therapy output may be modified to increase amplitude as it may be recognized that electroporation has not yet begun due to insufficient energy.
In another example, voltage measurements may be taken between therapy output bursts to observe apposition between bursts throughout the treatment cycle. For example, such measurements may indicate how much electrical capacitance there is in the target tissue; larger residual voltage indicates more capacitance, meaning cells are likely intact. As cells are porated, the capacitance may drop, yielding a lower voltage measurement between bursts and indicating therapy progress. In another example, inter-burst voltage measurement may be useful to help identify any action potentials generated by neural or muscle tissue during therapy delivery; excess action potentials can be indicative of possible muscle capture and, if detected, may be used to shorten pulse width, or identify a potential electrode interface charge imbalance, which can be corrected by the generation of counter pulses to remove such charge imbalance. The inter-burst voltage measurement may also be used to directly measure any electrode interface voltage, again to allow removal of such charge to reduce the likelihood of muscle activation.
The patient's cardiac signal can also be monitored, such as by capturing the surface or other ECG to provide timing information so that therapy windows are accurately timed relative to cardiac cycles. In still another example, the patient's cardiac signal may be monitored for changes, such an increase in heart rate, which may indicate changes in sympathetic tone and possible neural or muscle activation. The system may respond by attempting to remove any residual voltage on the electrode interfaces or reducing amplitude or pulse width, for example. The ECG may also be monitored to detect onset of any arrhythmia, from the relatively benign sinus tachycardia, to potentially harmful atrial fibrillation to deadly ventricular fibrillation, wherein the detection of onset can be used to interrupt therapy and trigger an alert or alarm.
A motion sensor, such as an accelerometer, may be placed on the patient and/or on or associated with a therapy probe/catheter to detect movement of the patient. Such motion may be indicative of the patient experiencing pain, and/or the electrical (or other) outputs of an ablation system causing muscle stimulation. The system may respond by attempting to remove any residual voltage on the electrode interfaces or reducing amplitude or pulse width, for example, or by communicating to a user/physician a need for repositioning or enhanced sedation, analgesia, or paralytics for the patient.
Muscle tone may be monitored by providing a strain sensor associated with one or more muscles or by placing sensing electrodes near a muscle group that has the potential to be stimulated or captured by the ablation therapy output; electrical signals captured using the sensing electrodes may be, for example, analog or digitally filtered through a passband to identify action potentials indicative of neural or muscle stimulus; a strain sensor may, alternatively, indicate muscle motion or preparation for motion.
Acoustic and/or optical sensors may be used, such as with provision at the end of a probe or on a separate instrument to identify, for example, tissue reactions to stimulus that are undesired, such as any acoustic output generated if tissue cells or interstitial fluid undergoes phase change; an optical sensor may identify flashing, for example, or may be used to capture a heat signal indicative of tissue heating in the vicinity of therapy delivery. In another example, a substance may be injected along with therapy delivery and observed using an optical sensor to detect whether and when the injected substance, such as a dye, drifts away from the target tissue, or undergoes a change due to expulsion of intracellular fluids in response to cell membrane rupture or electroporation. An acoustic sensor may also be used to detect heart sounds, which can in turn be used to aid in diagnosing arrhythmias and/or increased cardiac rate.
A blood pressure sensor may be used as well, whether internal to the patient or a wearable sensor such as a cuff, to detect changes in the patient's parasympathetic or sympathetic tone and/or vagal response, any of which can be indicative of rising stress and possible activation of neural or muscle tissue. Again, correction of any imbalance of charge between therapy delivery electrodes or reduction of pulse width, frequency, duty cycle, or amplitude, for example, may be used to reduce such activation.

The limits 212, 214 to be applied may be automatically generated by the ablation system control circuitry, for example, using information about the type of probe to be used, the type of tissue to be treated (i.e., whether small cell carcinoma is involve or a large cell tumor, as well as which patient anatomy is affected including lung, liver, brain, pancreas, stomach, etc.). Limits 212, 214 may be suggested or calculated by the system using measured information, such as by sensing the pre-ablation impedance between placed electrodes, or sensing other patient characteristics pre-therapy (blood pressure, heart rate, baseline electrical muscle noise, etc.) and implementing such sensed characteristics into a patient model that may further account for age, gender, weight, height and/or other patient characteristics. Such limits 212, 214 may instead be user generated. In some examples, the system provides suggested limits 212, 214 based on information entered by the user or calculated by the system itself. Where a therapy model or patient model is used, such models may be based on analytic approaches using, for example, a base model and building into the model variations based on the individual patient, or by reference to a database of actual patients, or a set of patient “types” in which a best match to the patient to be treated can be identified and relied upon for setting up an expected therapy regimen and response.

FIG. 4B builds further on the example of FIG. 4A by allowing the user to select a sense parameter 252 to be monitored and one or more limits 254, 256 which are then used to control a selectable output parameter as indicated at 258. More complex approaches may provide plural sense parameter inputs 252 that can be used to determine or modify a parameter of the output 258. In an example, in FIG. 4B, the sense parameter may be any of the above noted items, such as ECG signals, motion signals, temperature, blood pressure, acoustic or optical signals, strain sensor, voltage, current, resistance, impedance, complex impedance, etc., and may be used to control a parameter of an electrical output, including controlling any of current or voltage amplitude, peak or average power, energy delivered per unit time or per pulse, frequency, pulse width, burst duration, inter- or intra-burst periodicity, waveform shape, pulse shape, pulse segment shape or amplitude, therapy window duration, etc.

FIG. 4C shows another example. In this example the user can select from a list of therapy profiles 302. With a therapy profile selected, one or more limits or parameters to be monitored or controlled can be selected using boxes 304, 306, 308. In an example, the system selects the parameters at 304, 306, 308 and the user confirms them. In another example, the system will suggest the parameters. As noted, the parameters may be parameters used as inputs for controlling the output, or may be output parameters, or may be one or more of each.

In a first prophetic example, a therapy profile may be, for example, for treatment of a hepatic tumor using IRE in which the system monitors, for example, the parameters of impedance and temperature, to control output parameters of pulse width and amplitude. The therapy profile in this first prophetic example may establish a therapy regimen in which an initial, higher amplitude set of pulses are delivered to initiate cellular pore formation, leading to subsequent lower amplitude pulses (at various resolutions such as within the overall regimen, or within a therapy window, or within a burst). The therapy profile may follow a script for one or more pulses, bursts, or therapy windows, and then institute an adaptive method to provide still further control on the output parameters. The adaptive methods in the first prophetic example include increasing pulse width until a temperature change above a threshold minimum takes place, and decreasing pulse width if the sensed temperature change is above or trending toward a maximum, with the adaptive method further reducing amplitude over time if the impedance drops from an initial level, or increasing amplitude over time if impedance fails to drop in accordance with the expected course of the therapy profile.

In a second prophetic example, a large cell carcinoma therapy profile may make use of a thermal ablation technique for a first stage, and an IRE ablation technique for a second stage. For this second prophetic example, the sensed parameters may be used to transition from the first stage to the second stage as by, for example, monitoring for impedance drops that indicate completion of the first stage of the therapy, while also using an adaptive approach during each stage. In the second prophetic example, temperature sensing may be used in the first stage to manage temperature to a first, thermal ablation range while using acoustic sensors to detect any “popping” sounds indicative of non-linear responses (possibly including phase change which can indicate ablation that is or will become poorly controlled) during the first stage. Once impedance drops in an appropriate manner, the system switches to the second stage by reducing pulse width until the temperature drops to a desired range or below a (non-) thermal ablation threshold, at which point motion sensing is used to ensure that the IRE stage does not cause muscle contraction, with the adaptive sensing used to manage electrode interface polarization and/or to control pulse amplitude and pulse width. It may be noted that in some examples, the same accelerometer may be used to sense acoustics and motion with frequency selective filtering by applying a first, relatively higher frequency bandpass for acoustic sensing, and a second, relatively lower frequency bandpass for motion sensing. With this approach, as the pulse width is reduced, amplitude may be increased as temperature is monitored to ensure that the applied ablation targets electroporation rather than thermal ablation. A benefit of combined thermal and IRE ablation may include prompting immune response in the region of therapy to a greater extent than IRE alone, as IRE has in some studies been shown to prompt a lesser immune response than thermal ablation.

A third prophetic example comprise monitoring impedance changes during a therapy regimen to observe periods of reduced changes or plateaus in the impedance change. When such plateaus are observed, the adaptive system may add extended cycle delays (such as between 1 second and 5 minutes, or between 10-90 seconds) to allow tissue impedance to relax, recover and become electro-sensitized to additional burst trains. For example, tissue impedance may be allowed to recover up to 10% or more allowing subsequent trains to be more effective without adverse arc-over events. For example, with a burst train, one or more bursts in a train may be omitted, providing added relaxation of the tissue region during a therapy regimen.

A fourth prophetic example may comprise using a motion sensor, a sensor to identify electrical activity of a muscle (either pre-motive or as part of movement), or a sensor to detect neural action potentials, which allows a system monitor muscle contractions or electrical activity suggesting muscle contraction is about to occur or has nearly occurred. The system may, in response, adaptively change electrode pulsing (voltage, pulse width or pulse number) to reduce burst energy to relax, terminate or prevent contractions during an accumulated burst train—or to cycle electrode combination pairings to reduce stimulus using electrode combinations that can be temporally associated with movement or pre-movement.

Adaptive sensing may be used to control, for example, waveform type (biphasic, monophasic, multiphasic), waveform timing parameters (pulse width, pulse-pulse delay within a burst, delay between bursts, window for therapy), the quantity of pulses in a burst or the quantity of bursts to deliver overall or within a therapy window, rise time, fall time, slew rate, initial, average, or peak voltage or current, per pulse or per burst energy or power, average energy or power within a pulse, burst, or therapy window, and/or field density.

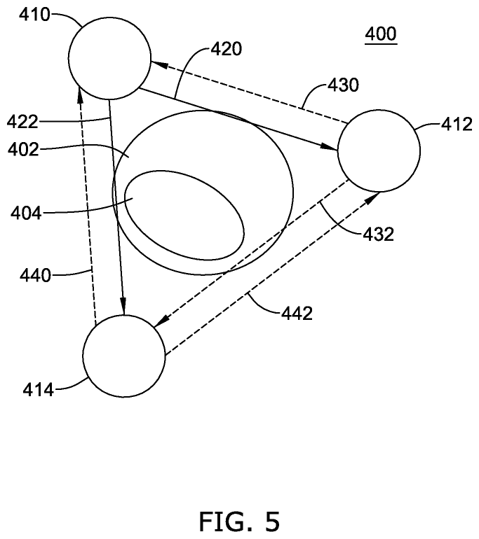

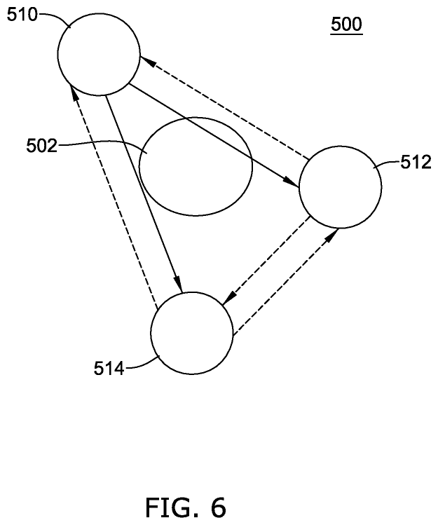

FIGS. 5-6 illustrate therapy application using a multiple electrode configuration and further illustrates how a planned therapy regimen may be modified adaptively during therapy output. Starting in FIG. 5, in a patient environment 400 a set of electrodes 410, 412, 414 are disposed about a therapy target 402. A three-electrode rotating therapy configuration is illustrated for exemplary and non-limiting purposes, as other electrode quantities and therapy patterns may be used in the present invention. In a first therapy step, electrode 410 is the cathode, issuing current toward anodes 412, 414 as indicated by lines 420, 422. A second step has electrode 412 as the cathode, issuing current toward anodes 410, 414, as indicated by lines 430, 432. A third step has electrode 414 as the cathode, issuing current toward anodes 410, 412, as indicated by lines 440, 442. Assuming the electrodes 410, 412, 414 form an equilateral triangle and the tissue is homogenous, one would expect that issuing equal voltage or currents in each step would provide a fairly balanced therapy, however, the triangle is not likely to be equilateral in practice, and the tissue may not be homogenous, and the actual target may not be perfectly central to the electrodes; for example, the target in fact may be the area defined at 404, rather than 402.

A therapy profile can be set up to adjust for the spatial nonconformity, and a system may use input from the user/physician to adjust the central target by controlling the voltages or currents through each electrode in an unequal way, steering the locus of stimulation to the desired target. As therapy is delivered an adaptive approach, such as highlighted above in FIG. 3C, can respond to changing conditions. For example, if temperature sensors are located near each of the three electrodes 410, 412, 414, the temperature of each electrode 410, 412, 414 can be managed by adjusting the voltage used when each electrode serves as anode or cathode, to direct more current through an electrode that is at a lower temperature or limit current to an electrode that is at a higher temperature. Such changes may be implemented within a pulse, within a burst, within a therapy window, or from one therapy window to the next.

FIG. 6 shows another example. Here, the electrodes and target tissue location are not symmetric as in FIG. 5, and the system may be configured to adjust both a therapy profile to account for the asymmetry, as well as using adaptive outputs to control therapy progress. For example, in FIG. 6, in the patient environment 500, the target tissue 502 lies closer to a line between electrodes 510 and 512, with electrode 514 somewhat distant. For current-controlled output, the following configuration may be used as the therapy profile:

| Step | 510 | 512 | 514 |
|---|---|---|---|
| 1 | +10 mA | −8 mA | −2 mA |
| 2 | −8 mA | +10 mA | −2 mA |
| 3 | −6 mA | −2 mA | +8 mA |

It can be seen that over time, the charge on the tissue interfaces of the three electrodes will not sum to zero. Thus, for example, a therapy regimen may include recovery periods, whether passive or active, to offset the buildup of charge on any given interface. Supposing a passive approach is used by shorting the electrodes together outside the therapy window, this may offset buildup of charge. A motion sensing device may be used to sense for any patient muscle movement responsive to residual charge on the electrodes, triggering an active recharge cycle in which charge is pumped to or from the individual electrodes to remove any offset. A temperature sensor may also be used to ensure that the temperature near electrode 510, which is most heavily used because it is closest to the target 502, remains within desired bounds. Finally, an electrical sensing apparatus may be used to sense for any myopotentials or action potentials that a charge imbalance can cause in the muscle or nerve tissue in the area, allowing corrective action to potentially be taken (again, to remove charge imbalance) before muscle movement even occurs. Thus, a multi-factorial sensing approach can be taken, with responses that vary by the type of sensed event takes place. In addition, as time goes on, the impedance between the electrode pair at 510, 512 would be expected to drop in response to the ablation; if no change is observed, amplitude, frequency of pulses in a burst or frequency of bursts, pulse width, or the number of pulses in a burst may be increased, if desired; other interelectrode impedances may not be as affected and so, for example, the impedance between electrodes 512 and 514 may be expected to not change; if it does, the system may take action to reduce current flows between those two electrodes. Thus, the adaptive feedback may be used as well to direct and/or control therapy.

In another example, with an asymmetric configuration as in FIG. 6, the system may adopt a combination approach in which a controlled voltage is issued between electrodes 510, 512, while a controlled current is issued at electrode 514, relative to either of electrodes 510, 512 or to a remote, indifferent electrode, for example.

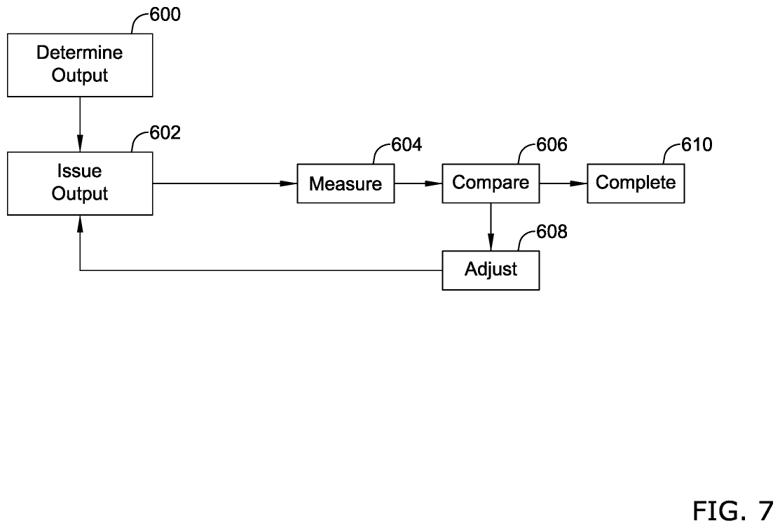

FIGS. 7-9 show illustrative methods in block form. The illustrative methods may also be implemented in apparatus form, for example, as a system having configurations for performing the steps and methods shown in each Figure.

Referring first to FIG. 7, a method for therapy delivery may comprise first determining an output or output parameter set, as indicated at 600. The operation at step 600 may provide an initial or first output parameters set for use in an overall therapy regiment, or for a first burst during a therapy window, or for one or more initial pulses in a burst, or for a first portion of a therapy pulse, in each of several different levels of resolution. In some examples, the step of determining an output comprises receiving a set of parameters for therapy delivery from a physician or user, such as by receiving the parameters directly, or by the physician or user selecting a predetermined program having specified parameters, wherein the predetermined program may automatically generate initial or first parameters, or may be operable to receive one or more patient or therapy target characteristics for purposes of tailoring the program (i.e., entering patient weight, target size, or tissue type, and allowing a program analytics tool to generate parameters).

Also in the method, steps as illustrated above in FIGS. 4A-4C may be used as preparation, to thereby identify therapy parameters and to allow the user to select feedback inputs. For example, block 600 may comprise a preparation step including providing the user a list of available closed loop control parameters to select from, and receiving from the user a selection of one or more closed loop control parameters. An additional preparation step may be, for at least one user selected closed loop parameter, presenting the user with an input screen for selecting or approving one or more limits for the user selected closed loop parameter, again as discussed above relative to FIGS. 4A-4C. The closed loop control parameters may include or take the form of any of the feedback parameters described above.

Next, the method comprises a signal generator delivering a therapy regimen by issuing output, as indicated at 602, including, in a first pass, generating a first electrical output having a first output parameter set. The method comprises sensing a signal to measure one or more of the selected closed loop control parameters, as indicated at 604. The method next includes comparing the sensed signal to the user selected or approved limit for the user selected closed loop parameter, as indicated at 606. A therapy loop then takes place, in which the next step is to adjust the first output parameter set to create a second output parameter set, as indicated at 608, followed by returning to block 602 and generating a second electrical output using the second output parameter set. Adjustments at 608 may include changing current, voltage, power, energy, pulse width, frequency, repetition rate, burst rate, the number of pulses within a burst, pulse shape, pulse segment shape or amplitude, slew, electrode selection (either in terms of which electrodes are used or in terms of which electrodes are anodes or cathodes), pulse or therapy type (monophasic, biphasic, monopolar, bipolar, current- or voltage-controlled) and other features noted previously.

In the method, one or more of the parameter sets used is configured for ablating tissue, for example, by having the initial parameter set configured for ablation as well as subsequent parameter sets. An initial parameter set or a subsequent parameter set may also or instead be a non-therapy parameter set, which may be used from time to time in order to, for example, reduce local temperature, allow measurement of the patient's intrinsic status, and/or correct charge imbalances on the electrode-tissue interfaces. In some examples, the initial parameter set may not be configured for ablation, and may instead be operable to allow the system to deliver sub-therapy level pulses or bursts to determine electrode configuration, impedances, or other characteristics, such as baselines for any feedback parameters prior to ramping power to an elevated level for ablation purposes.

An iterative process follows, with the method repeating a cycle of blocks 602, 604, 606, 608, until it is determined that therapy is complete, as indicated at 610. Therapy complete may be based on sensed characteristics, if desired, or may be determined based on a quantity of pulses or bursts delivered, or a period of time, in other examples, without limiting therapy complete declaration to these specific rationales. The cycle 602, 604, 606, 608 may take place within a pulse using pulse segments as shown above in FIG. 3B, or may occur across a burst of pulses as also shown in FIG. 3C, or may take place from one burst to the next in a therapy regimen, or may be performed to define a therapy for a therapy window following completion of a first therapy window.

Feedback parameters that may be measured and compared at blocks 604, 606 may include, for example and without limitation, the list provided above. In some examples, peak or average voltage, current, energy, power, temperature, or other measurable parameter may be a feedback parameter. Phase measurement may be a feedback parameter, as well as reactive impedance, interpulse or interburst voltage measurement, and/or multi-path impedance.

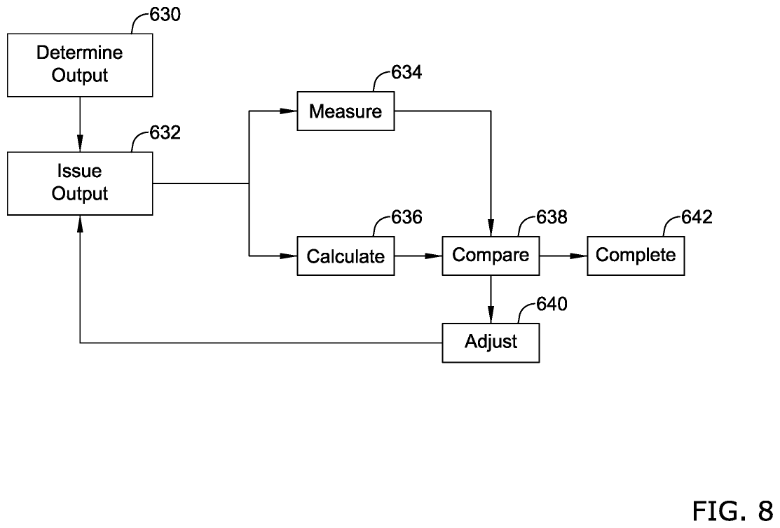

FIG. 8 shows another illustrative example. Here the method again begins with the determination of an output at 630, which may be an initial output similar to block 600, above. The output is then issued as indicated at 632. During or after issuance of the output, one or more measurements are taken as indicated at 634. Such measurements may include, for example and without limitation, the capture of any of the noted feedback parameters described above. An expected value or range of values for the measurements is also calculated, as indicated at 636. For example, the calculated value may relate to the expected changes in tissue characteristics that a therapy profile can be used to calculate. For example, given an initial or prior sensed impedance between two electrodes, as therapy is delivered it may be expected that the impedance would change, and the calculated value or range of values at 636 would reflect an expected change in impedance in response to delivered therapy; such measurements and calculations can be iteratively repeated during the therapy regimen. In another example, given an initial or prior sensed temperature, an expected temperature can be calculated; for a thermal ablation therapy the temperature may be expected to increase above normal body temperature to a thermal ablation range; for a non-thermal ablation therapy such as IRE, no change or a lesser temperature change can be expected. In another example, as cells in a target tissue region are destroyed, measured voltage in the area during a time between pulses, between bursts, or between therapy windows, may be expected to drop as cellular electrical activity ceases due to cell death, thus a prior measurement can be compared to a later measurement, with the calculation at 636 applying a model for changes in the measured voltage. In another example, if patient motion is observed, or cell action potentials are observed, changes to therapy output may include issuing a non-therapy output to reduce any electrode interface charges, or to reduce amplitude or pulse width to prevent muscle stimulation during therapy.

At 638 the measured feedback parameter is compared to the calculated expected range or value. The result of the comparison at 638 is used to determine adjustments to the therapy at 640. Adjustments at 640 may include, without limitation, changing current, voltage, power, energy, pulse width, frequency, repetition rate, burst rate, the number of pulses within a burst, pulse shape, pulse segment shape or amplitude, slew, electrode selection (such as, for example and without limitation, changing which electrodes are used as anodes, cathodes or indifferent electrodes and/or which electrodes are unused), pulse or therapy type (monophasic, biphasic, monopolar, bipolar, current- or voltage-controlled) and other features noted previously. For example, if temperature is trending higher than expected, pulse width, frequency, amplitude or duty cycle of therapy may be reduced; if impedance is not dropping as predicted, therapy duration may be extended or therapy parameters may be modified to increase energy delivery. In another example, if the comparison at 638 shows expected therapy progress toward completion, other changes can be made to, for example, switch from a thermal output to a non-thermal ablation output, where the non-thermal ablation output may be used to ensure that tract seeding is prevented.

As with FIG. 7, a therapy loop is entered, including blocks 632, 634, 636, 638 and 640, as measurements are made, therapy profile predictions are calculated, and the actual and expected outcomes are compared. The cycle may occur at various levels of resolution, including within a single therapy pulse, within a burst of therapy pulses, by defining one pulse based on measurements taken during a prior pulse, from one burst to the next in a therapy regimen, or from one therapy window to the next, for example. In other examples, the cycle shown may be periodic, such as occurring once a second, or every ten seconds, or at some other period during a therapy regimen.

The cycle can continue until therapy complete is declared, at 642. Therapy complete may be based on sensed characteristics, if desired, or may be determined based on a quantity of pulses or bursts delivered, or a period of time, in other examples, without limiting therapy complete declaration to these specific rationales. Thus the example of FIG. 8 shows a therapy system operation comprising configuring a first output therapy parameter set using the selected available therapy profile, generating one or more first therapy outputs using the first output therapy parameter set, sensing one or more first feedback parameters, comparing the first feedback parameters to an expected feedback parameter to generate one or more first comparison results, wherein the expected feedback parameter is associated with the selected therapy profile, and configuring a second output therapy parameter set using the first comparison results.

The feedback parameters may comprise any of those listed above, including, for example and without limitation, a relative change in impedance, a change in sensed phase, a measurement of inter-pulse or inter-burst voltage, a measurement of multi-path impedance or impedance changes. In the example, the therapy profile used at 636, as well as possibly used to determine an initial or prior output at 630, may comprise a schedule of therapy output parameters to use during the duration of the therapy regimen, wherein the signal generator is adapted to configure the second output therapy parameter set as follows: if the first feedback parameters correlate with the expected feedback parameters, using the schedule of therapy output parameters for the selected therapy profile to define the second output therapy parameter set; or if the first feedback parameters do not correlate with the expected feedback parameters, modifying the schedule of therapy output parameters for the selected therapy profile in response to the first feedback parameters.

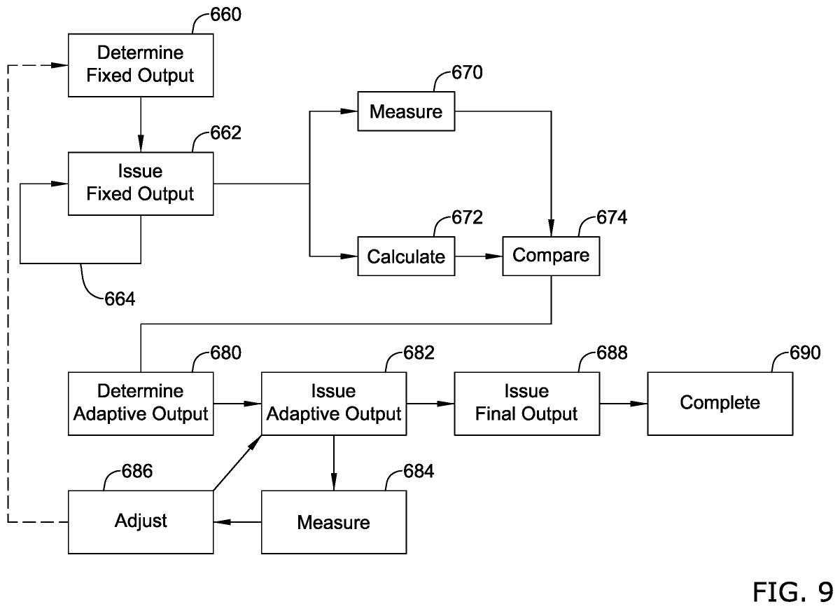

FIG. 9 shows another illustrative method. Here, the method has (at least) two general phases for therapy delivery, including fixed outputs determined according to a schedule, and adaptive outputs that are determined using one or more sensed feedback parameters. The example starts illustratively with a fixed output portion, and then proceeds to an adaptive output, followed by optional final outputs; the steps/stages may be performed in different order.

In the example, a fixed output is determined at 660, and then issued as indicated at 662, with looping back to the fixed outputs 662 as shown at 664. The fixed output may be calculated using a selected therapy profile or may be entered, for example, by a user. The fixed outputs at 660/662 can be identical to one another or may vary from one another according to a therapy profile, or therapy schedule; either way, the outputs at 662 are generated without reference to a feedback parameter or other adaptive method. After some predetermined period, or in response to an event, such as a sensed temperature or current out of range or meeting some threshold, the method proceeds to a measurement 670 of a feedback parameter, in parallel with calculating or determining (such as by a lookup function) 672 an expected value or range for the feedback parameter, leading to a comparison at 674 that is used to start an adaptive output cycle. In the adaptive output cycle, as indicated at 680, the adaptive output is determined, then issued at 682, and a measurement is taken at 684, leading to adjustment 686, returning issuance of the adaptive output at 682. The adaptive cycle may be exited with return to a fixed output, if desired and a shown by the dashed line.

In other examples, optionally, a set of final outputs may be issued at 688. In an example, the adaptive and fixed output loops are used to perform ablation in the main, while the final outputs 688 are directed to finishing the procedure by issuing outputs that prevent tract seeding prior to or during removal of a therapy probe. Following the optional final outputs at 688, the procedure is deemed complete at 690. The feedback parameters to use, as well as the adjustments that can be made, may encompass those described above relive to FIGS. 7-8. The example shown may be described as, for example, using a user-selected therapy profile to defining, for the selected therapy profile, at least a portion of a therapy regimen comprising a plurality of bursts of pulses, each burst comprising a plurality of pulses, each pulse comprising a plurality of pulse segments; configuring a first output therapy parameter set using the selected available therapy profile, the first output therapy parameter set defining a first predetermined segment of a predetermined pulse of a predetermined burst; generating the first predetermined segment of using the first output therapy parameters; sensing one or more first feedback parameters; using the sensed first feedback parameters to define a second predetermined segment occurring after the first predetermined segment in the predetermined pulse of the predetermined burst. In other examples, the method of FIG. 9 may be used at other resolutions, such as to define pulses within a burst, or bursts within a series of bursts or in a therapy window, or therapy from one window to the next.

While several of the above examples reference therapy delivery using therapy windows, such as windows defined relative to the patient's cardiac cycle, other examples omit the windows and deliver therapy without reference to a patient's cardiac cycle or other biological phenomenon. Additionally, while therapy bursts are described in several examples, it may be the case that therapy is simply delivered at a fixed frequency, rather than using bursts in which pulses are delivered at a first period with a quiescent period between bursts of a second, longer period.

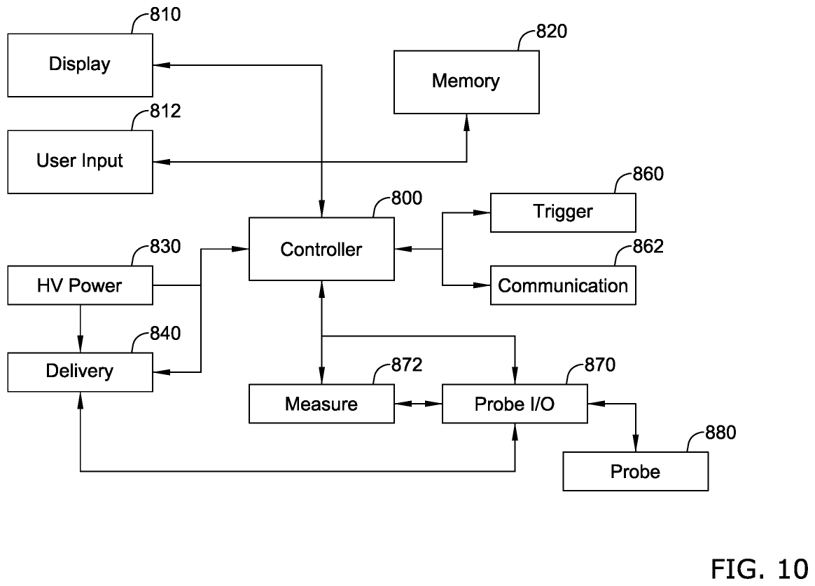

FIG. 10 is a block diagram for an illustrative ablation system. The system of FIG. 10 may implement the methods shown above in the preceding figures. The apparatus contains a controller block 800 which may include, for example, a state machine, a microcontroller or microprocessor adapted to execute programmable instructions, which may be stored in a memory 820 that can also be used to store history, events, parameters, sensed conditions, alerts, and a wide variety of data such as template programs, information related to probes 880, and the like. The memory 820 may include both volatile and non-volatile memory types, and may include a port for coupling to a removeable memory element such as an SD card or thumb drive using a USB port. The memory 820 may contain executable instruction sets or other data that will allow the system to present to a user options for feedback parameters and therapy profiles as described above, including therapy generating instructions that can use a therapy profile and, optionally, one or more patient conditions or characteristics to determine therapy parameters and expected therapy progressions. In some examples, a remote database or other memory structure may be accessed using the communication block 862 that is further described below to obtain, for example, therapy profiles from a central repository or other storage location, such as via the Internet.

The controller block 800 is coupled to a display 810 and user input 812. The display 810 and user input 812 may be integrated with one another by including a touchscreen. The display 810 may be a computer screen and/or touchscreen and may also include lights and speakers to provide additional output statuses or commands, verbal prompts, etc. The user input 812 may include one or more of a keyboard, a mouse, a trackball, a touchpad, a microphone, a camera, etc. Any inputs by the user may be operated on by the controller block 800.

The controller block 800 may include, for example, and without limitation, a microcontroller, a microprocessor, a state machine, etc. The controller block 800 may also include, alone or in association with a microcontroller, microprocessor or state machine, one or more application specific integrated circuits (ASICs) to provide additional functionality, such as an ASIC for filtering and analyzing an ECG to identify therapy windows, or analog to digital conversion circuits for handling received signals from a probe apparatus. An ASIC may include sample/hold circuitry, a digital signal processor (DSP), a digital filter subcircuit, and other circuitry as needed.

The controller block 800 is also coupled to an HV Power block 830, which may comprise a capacitor stack or other power storage apparatus, coupled to a charger or voltage multiplier that provides a step up from standard wall power voltages to very high powers, in the hundreds to thousands of volts. A therapy delivery block 840 is shown as well and may include high power switches arranges in various ways to route high voltages or currents from the HV power 830 to a probe input/output (Probe I/O) 870, which in turn couples to a probe 880. In some examples, the HV power block 830 and Delivery block 840 may incorporate circuitry and methods described in U.S. Provisional Patent Application No. 62/819,101, filed Mar. 15, 2019 and titled WAVEFORM GENERATOR AND CONTROL FOR SELECTIVE CELL ABLATION, the disclosure of which is incorporated herein by reference. The HV Power block 830 and Delivery Block 840 may be used to provide voltage-controlled and/or current-controlled outputs. For example, the use of a digital to analog convertor and an associated amplifier may be used to generate a controlled voltage output. Also, various H-bridge topologies are known for use in the delivery circuit block 840 (such as shown in U.S. Pat. Nos. 6,952,608 and/or 7,555,333, the disclosures of which are incorporated herein by reference) to provide a current-controlled output from an HV capacitor stack. Other control circuits, such as a current mirror circuit (with a current multiplier if needed) can be used to provide a controlled current. Thus there may be different configurations of the same delivery circuit block 840 and/or HV power block 830 to provide current-versus voltage-controlled output, or there may be separate circuitry. Given that the system does not face significant size constraints (it would not typically be implantable in these examples), the use of separate dedicated circuits for each of current control and voltage control in the delivery block 840 is likely.

The Probe I/O 870 may include a smart probe interface that allows it to automatically identify the probe 880 using an optical reader interface (barcode or QR code) or using an RFID chip that can be read via an RF reader, or a microchip that can be read once the probe 880 is electrically coupled to a port on the Probe I/O 870. A measuring circuit 872 is coupled to the Probe I/O 870, and may be used to measure voltages, currents and/or impedances related to the probe, such as measuring the current flowing through a connection to the probe 880, or the voltage at an output of the Probe I/O 870. The Probe I/O may comprise electrical couplings to the Probe 880 for purposes of therapy delivery, or for sensing/measurement of signals from the Probe 880, using for example sensing electrodes or sensing transducers (motion, sound, vibration, temperature or optical transducers, for example), as well as an optical I/O if desired to allow the output or receipt of optical energy, such as using optical interrogation of tissue or issuing light at therapeutic levels or even at ablation power levels. Not all of these options are required or included in some embodiments.

The controller block 800 is also coupled to trigger circuitry 860 and/or communications circuitry 862. The trigger circuitry may include, for example, an ECG coupling port that is adapted to receive electrodes or an ECG lead system for capturing a surface ECG or other signal from the patient for use in a triggered therapy mode. A communications circuit 862 may instead be used to wirelessly obtain a trigger signal, either a trigger that is generated externally, or a raw signal (such as an ECG) to be analyzed internally by the controller 800. The communication circuit 862 may include a transceiver having one or more of Bluetooth or WIFI antennas and driver circuitry to wirelessly communicate status, data, commands, etc. before, during or after therapy regimens are performed. If desired, the trigger 860 may have a dedicated transceiver itself, rather than relying on the system communication block 862. The communication block 862 may also be used to obtain data remotely, such as from a database or other repository, to provide therapy profiles, for example, if desired, or to offload data related to therapy that has been delivered to allow updating of any such remote database or repository of information. As noted, the controller block 800 may include an ASIC; if so, the trigger and communication circuitry may be partly or wholly included as part of the ASIC, if desired.

The probe 880 may take any suitable form, such as a LeVeen® needle, or a probe as shown in U.S. Pat. Nos. 5,855,576, 6,638,277, and/or US PG Pat. Pub. No. 2019/0223943, the disclosure of which is incorporated herein by reference, or other suitable ablation designs such as using multiple probes each comprising a needle electrode, either integrated into one structure or separately placed. The probe 880 may include one or more indifferent or return electrodes, such as plates that can be cutaneously placed. A separately placed cutaneous electrode, for use as an indifferent, return electrode, or as an anode or cathode if desired, may be placed separate from the probe 880, if desired.

Each of the following non-limiting embodiments can stand on its own, or can be combined in various permutations or combinations with one or more of the other examples or embodiments described above or below. Citations to reference numbers below should be understood as further encompassing reference to above text that describes the blocks, designs, or features each such reference number relates to.

A first illustrative and non-limiting embodiment comprises a system for controlling an ablation therapy comprising: a signal generator (FIG. 2, 108 and as shown in FIG. 10) adapted to provide electrical output for ablation therapy; a probe (FIG. 2, 100 and FIG. 10, 880) for delivering therapy generated by the signal generator to a patient; and a user interface (FIG. 2, 120, FIGS. 4A-4C, and FIG. 10 at 810) operatively linked to the signal generator to provide data to a user related to therapy, and to receive commands from a user; the improvement comprising: the user interface being configured to interact with a user with: selection means (FIG. 4A, 210 and FIG. 4B, 252) adapted to provide the user a list of available closed loop control parameters to select from and receive from the user a selection of one or more closed loop control parameters; and limit setting means (FIG. 4A, 212, 214, and FIG. 5B 254, 256) adapted to, for at least one user selected closed loop parameter, present the user with an input screen for selecting or approving one or more limits for the user selected closed loop parameter and receive the user's selection or approval of the one or more limits of the user selected closed loop parameters.

Further in the first illustrative and non-limiting embodiment, the signal generator being configured to deliver a therapy regimen with: determining means (operating as indicated in FIG. 7, 600, FIG. 8, 630, FIG. 9 660, 680, for example as implemented in FIG. 10, 800, where the controller block performs various calculations) configured to determine a first output parameter set for therapy delivery; generating means (operating as indicated in FIG. 7, 602, FIG. 8, 632, FIG. 9, 662, 682, for example as implemented in FIG. 10, 830, 840, 870) to issue therapy signals according to output parameter sets; measuring means (operating as indicated in FIG. 7, 604, FIG. 8, 634, FIG. 9, 670, 684, for example as implemented in FIG. 10, 872) for sensing and measuring a signal related to the user selected closed loop parameter from the selection means; comparing means (operating as indicated in FIG. 7, 606, FIG. 8, 638, FIG. 9, 674, for example as implemented in FIG. 10, 800) for comparing the measured signal from the measuring means to the user selected or approved limit for the user selected closed loop parameter from the setting means; and adjusting means (operating as indicated in FIG. 7, 608, FIG. 8, 640, FIG. 9, 686, for example as implemented in FIG. 10, 800 acting in concert with 830, 840, and/or 870) for making adjustments to an output parameter set responsive to the comparing means; wherein the signal generator is configured to deliver a first portion of a therapy regimen using the first output parameter set and to then rely upon the measuring means, comparing means, and adjusting means to modify output parameters for subsequent portions of the therapy regimen (such adjustments are illustratively shown in FIGS. 3B-3C, and described in associated with other figures including FIGS. 5-9).

Additionally or alternatively to the first illustrative and non-limiting embodiment, the closed loop control parameters may comprise at least user selectable options for phase. Additionally or alternatively to the first illustrative and non-limiting embodiment, the closed loop control parameters may comprise at least user selectable options for reactive impedance.

Additionally or alternatively to the first illustrative and non-limiting embodiment, the closed loop control parameters may comprise at least user selectable options for inter-pulse or inter-burst voltage.

Additionally or alternatively to the first illustrative and non-limiting embodiment, the closed loop control parameters may comprise at least user selectable options for multi-path impedance.

Additionally or alternatively to the first illustrative and non-limiting embodiment, the therapy regimen may comprise a plurality of bursts each comprising a plurality of pulses, wherein the signal generator is configured to adjust therapy parameters within a single pulse, and/or wherein the signal generator is configured to adjust therapy parameters from one pulse to the next within the same burst.

Additionally or alternatively to the first illustrative and non-limiting embodiment, the probe comprises at least three electrodes each of which is independently selectable relative to other electrodes (as shown in FIG. 2); the generating means uses the output parameter sets to select among the at least three electrodes which to use as anode, cathode, indifferent or not connected when issuing therapy; and the adjusting means is configured to modify output parameters to change which of the electrodes are used as anode, cathode, indifferent or not connected.

Additionally or alternatively to the first illustrative and non-limiting embodiment, the system may comprise a return electrode separate from the probe, wherein: the probe comprises at least two electrodes each of which is independently selectable; the generating means uses the output parameter sets to select among the return electrode and the at least two probe electrodes which to use as anode, cathode, indifferent or not connected when issuing therapy; and the adjusting means is configured to modify output parameters to change which of the return electrode and the at least two probe electrodes are used as anode, cathode, indifferent or not connected.

Additionally or alternatively to the first illustrative and non-limiting embodiment, the generating means may be configured with an adjustable slew rate and the adjusting means is configured to modify output parameters to change slew rate used by the generating means.

Additionally or alternatively to the first illustrative and non-limiting embodiment, the user interface comprises program means (FIG. 4C at 302) adapted to provide the user with a selectable set of therapy profiles and receive a selection of a therapy profile, and the determining means is configured to determine the first output parameter set using the selected therapy profile. Additionally or alternatively, the comparing means is configured to adjust the user selected or approved limit for the user selected closed loop parameter in accordance with expected values according to the user selected therapy profile, thereby adjusting therapy according to the user selected therapy profile.

Additionally or alternatively to the first illustrative and non-limiting embodiment wherein the user interface comprises therapy parameter receiving means to receive initial parameters for therapy from the user, and the determining means is configured to determine the first output parameter set using the initial parameters entered by the user (such utility is described relative to FIG. 7 at 600, FIG. 8 at 632, and FIG. 9 at 660, and may be implemented on a touchscreen or keyboard-type input relative to the user interface 120 in FIG. 2 and/or display 810 and user input 812 in FIG. 10).

A second illustrative and non-limiting embodiment takes the form of a system for controlling an ablation therapy comprising: a signal generator (FIG. 2 at 108, and generally shown in FIG. 10) adapted to provide electrical output for ablation therapy; a probe (FIG. 2 at 100 and FIG. 10 at 880) for delivering therapy generated by the signal generator to a patient; and a user interface (FIG. 2 at 120 and FIG. 10 at 810/812) operatively linked to the signal generator to provide data to a user related to therapy, and to receive commands from a user; the improvement comprising: the user interface comprising a therapy profile selection means (FIG. 4C at 302) for allowing the user to select among at least first and second regimens for therapy to the patient; the signal generator comprising: therapy parameter defining means (operating as indicated in FIG. 7, 600, FIG. 8, 630, FIG. 9 660, 680, for example as implemented in FIG. 10, 800, where the controller block performs various calculations) for determining therapy parameters responsive to the user selected therapy profile; generating means (operating as indicated in FIG. 7, 602, FIG. 8, 632, FIG. 9, 662, 682, for example as implemented in FIG. 10, 830, 840, 870) for issuing therapy pulses according to therapy parameters received from the therapy parameter defining means; feedback means (operating as indicated in FIG. 7, 604, FIG. 8, 634, FIG. 9, 670, 684, for example as implemented in FIG. 10, 872) for quantifying therapy feedback parameters; calculating means (operating as indicated in FIG. 8 at 636 and FIG. 9 at 672) for determining expected values for the therapy feedback parameters using the user selected therapy profile; comparing means (operating as indicated in FIG. 7, 606, FIG. 8, 638, FIG. 9, 674, for example as implemented in FIG. 10, 800) for comparing the quantified therapy feedback parameters to the expected values for the therapy feedback parameters; and adjusting means (operating as indicated in FIG. 7, 608, FIG. 8, 640, FIG. 9, 686, for example as implemented in FIG. 10, 800 acting in concert with 830, 840, and/or 870) for adjusting operation of the therapy defining means responsive to comparisons by the comparing means.

Additionally or alternatively to the second illustrative and non-limiting embodiment, the feedback means may monitor, without limitation, phase, reactive impedance, inter-pulse or inter-burst voltage, and/or multi-path impedance as the therapy feedback parameter(s).

Additionally or alternatively to the second illustrative and non-limiting embodiment, the therapy profile may be used to implement a therapy regimen having a plurality of bursts each comprising a plurality of pulses, wherein the signal generator is configured to adjust therapy parameters within a single pulse, and/or wherein the signal generator is configured to adjust therapy parameters from one pulse to the next within the same burst.

Additionally or alternatively to the second illustrative and non-limiting embodiment, the probe comprises at least three electrodes each of which is independently selectable relative to other electrodes (as shown in FIG. 2); the generating means is configured to select among the at least three electrodes which to use as anode, cathode, indifferent or not connected when issuing therapy; and the adjusting means is configured to modify output parameters to change which of the electrodes are used as anode, cathode, indifferent or not connected.

Additionally or alternatively to the second illustrative and non-limiting embodiment, the system may comprise a return electrode separate from the probe, wherein: the probe comprises at least two electrodes each of which is independently selectable; the generating means uses the output parameter sets to select among the return electrode and the at least two probe electrodes which to use as anode, cathode, indifferent or not connected when issuing therapy; and the adjusting means is configured to modify output parameters to change which of the return electrode and the at least two probe electrodes are used as anode, cathode, indifferent or not connected.

Additionally or alternatively to the second illustrative and non-limiting embodiment, the generating means may be configured with an adjustable slew rate and the adjusting means is configured to change slew rate used by the generating means.

Additionally or alternatively to the first illustrative and non-limiting embodiment wherein the user interface comprises therapy parameter receiving means to receive initial parameters for therapy from the user, and the determining means is configured to determine the first output parameter set using the initial parameters entered by the user (such utility is described relative to FIG. 7 at 600, FIG. 8 at 632, and FIG. 9 at 660, and may be implemented on a touchscreen or keyboard-type input relative to the user interface 120 in FIG. 2 and/or display 810 and user input 812 in FIG. 10).

A third illustrative and non-limiting embodiment takes the form of a system for controlling an ablation therapy comprising: a signal generator (FIG. 2, 108 and as shown in FIG. 10) adapted to provide electrical output for ablation therapy and having sensing means (noted as a measuring block 872 in FIG. 10, for example, but also receivable via the communication block 862 in FIG. 10) for sensing one or more parameters of at least one of the electrical output or a patient and first generating means to generate a controlled voltage output, and second generating means to generate a controlled current output (each generating means represented as a different configuration of the HV power block 830 and delivery block 840 in FIG. 10 or, in the alternative, as separate circuits within the delivery block 840); a probe (FIG. 2, 100 and FIG. 10, 880) for delivering therapy generated by the signal generator to a patient; and a user interface (FIG. 2, 120, FIGS. 4A-4C, and FIG. 10 at 810) operatively linked to the signal generator to provide data to a user related to therapy, and to receive commands from a user; the improvement comprising: wherein the signal generator is configured to: at a first time, use the first generating means to generate a voltage-controlled therapy output for delivery to the patient with the probe; at a second time, use the sensing means to sense a change in a sensed parameter and, responsive thereto, switch to using the second generating means to thereby generate a current-controlled therapy output for delivery to the patient with the probe (such an operation is described in relation to FIG. 3B as an adaptive approach).

Additionally or alternatively to the third illustrative and non-limiting embodiment, the voltage-controlled output is generated initially while the sensing means is used to monitor current flow and temperature. Additionally or alternatively the system may comprise adjusting means to adjust parameters of therapy delivery in response to sensed patient conditions or therapy parameters, including manipulating the controlled voltage to place current flow in a target range until a target temperature is reached, at which point the system switches to using the second generating means.

Additionally or alternatively to the third illustrative and non-limiting embodiment wherein the user interface comprises therapy parameter receiving means to receive initial parameters for therapy from the user, and the determining means is configured to determine the first output parameter set using the initial parameters entered by the user (such utility is described relative to FIG. 7 at 600, FIG. 8 at 632, and FIG. 9 at 660, and may be implemented on a touchscreen or keyboard-type input relative to the user interface 120 in FIG. 2 and/or display 810 and user input 812 in FIG. 10).

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A system for controlling an ablation therapy comprising:
a signal generator adapted to provide electrical output for ablation therapy; and
a user interface operatively linked to the signal generator, the user interface configured to interact with a user by:
providing the user a list of available closed loop control parameters to select from;
receiving from the user a selection of one or more closed loop control parameters; and
for at least one user selected closed loop parameter, presenting the user with an input screen for selecting or approving one or more limits for the user selected closed loop parameter;
further wherein the signal generator is configured to deliver a therapy regimen as follows:
generating a first electrical output having a first output parameter set;
sensing a signal related to the user selected closed loop parameter and comparing the sensed signal to the user selected or approved limit for the user selected closed loop parameter;
adjusting the first output parameter set to create a second output parameter set; and
generating a second electrical output using the second output parameter set, wherein at least the second output parameter set is configured for ablating tissue.

2. The system of claim 1 wherein the closed loop control parameters comprise at least user selectable options for phase.

3. The system of claim 1 wherein the closed loop control parameters comprise at least user selectable options for reactive impedance.

4. The system of claim 1 wherein the closed loop control parameters comprise at least user selectable options for inter-pulse or inter-burst voltage.

5. The system of claim 1 wherein the closed loop control parameters comprise at least user selectable options for multi-path impedance.

6. The system of claim 1 wherein the therapy regimen comprises a plurality of bursts each comprising a plurality of pulses, wherein the first and second electrical outputs occur within the same pulse.

7. The system of claim 1 wherein the therapy regimen comprises a plurality of bursts each comprising a plurality of pulses, wherein the first and second electrical outputs occur within separate pulses of the same burst.

8. The system of claim 1 wherein the first and second electrical outputs differ from one another in terms of electrodes selected as anodes or cathodes for each of the outputs.

9. The system of claim 1 wherein the first and second electrical outputs differ from one another in terms of slew rate.

10. A system for controlling an ablation therapy comprising:
a signal generator adapted to provide electrical output for ablation therapy; and
a user interface operatively linked to the signal generator, the user interface configured to interact with a user by:
providing the user a list of available therapy profiles to select from; and
receiving from the user a selection of one of the available therapy profiles;
further wherein the signal generator is configured to deliver a therapy regimen as follows:
configuring a first output therapy parameter set using the selected therapy profile;
generating one or more first therapy outputs using the first output therapy parameter set;
sensing one or more first feedback parameters;
comparing the first feedback parameters to an expected feedback parameter to generate one or more first comparison results, wherein the expected feedback parameter is associated with the selected therapy profile; and
configuring a second output therapy parameter set using the first comparison results.

11. The system of claim 10 wherein the first feedback parameters and the expected feedback parameter relate to a relative change in impedance.

12. The system of claim 10 wherein the first feedback parameters and the expected feedback parameter relate to a change in sensed phase.

13. The system of claim 10 wherein the first feedback parameters and the expected feedback parameter relate to inter-pulse or inter-burst voltage.

14. The system of claim 10 wherein the first feedback parameters and the expected feedback parameter relate to multi-path impedance.

15. The system of claim 10 wherein the therapy regimen comprises a plurality of bursts each comprising a plurality of pulses, wherein the first and second electrical outputs occur within the same pulse.

16. The system of claim 10 wherein the therapy regimen comprises a plurality of bursts each comprising a plurality of pulses, wherein the first and second electrical outputs occur within separate pulses of the same burst.

17. The system of claim 10 wherein the therapy profiles comprise a schedule of therapy output parameters to use during the duration of the therapy regimen, wherein the signal generator is adapted to configure the second output therapy parameter set as follows:
if the first feedback parameters correlate with the expected feedback parameters, using the schedule of therapy output parameters for the selected therapy profile to define the second output therapy parameter set; or
if the first feedback parameters do not correlate with the expected feedback parameters, modifying the schedule of therapy output parameters for the selected therapy profile in response to the first feedback parameters.

18. The system of claim 10 wherein the first and second electrical outputs differ from one another in terms of electrodes selected as anodes or cathodes for each of the outputs.

19. The system of claim 10 wherein the first and second electrical outputs differ from one another in terms of slew rate.

20. A system for controlling an ablation therapy comprising:
a signal generator adapted to provide electrical output for ablation therapy; and
a user interface operatively linked to the signal generator, the user interface configured to interact with a user by:
providing the user a list of available therapy profiles to select from; and
receiving from the user a selection of one of the available therapy profiles;
further wherein the signal generator is configured to deliver a therapy regimen as follows:
defining, for the selected therapy profile, at least a portion of a therapy regimen comprising a plurality of bursts of pulses, each burst comprising a plurality of pulses, each pulse comprising a plurality of pulse segments;
configuring a first output therapy parameter set using the selected available therapy profile, the first output therapy parameter set defining a first predetermined segment of a predetermined pulse of a predetermined burst;
generating the first predetermined segment of using the first output therapy parameters;
sensing one or more first feedback parameters;
using the sensed first feedback parameters to define a second predetermined segment occurring after the first predetermined segment in the predetermined pulse of the predetermined burst.
